(19) United States
(12) Patent Application Publication
(10) Pub. No.: US 2005/0118721 A1
(43) Pub. Date: Jun. 2, 2005

(54) Identification of cell differentiation states

(76) Inventors: Sven Olek, Berlin (DE); Alexander Olek, Berlin (DE)

Correspondence Address: DAVIS WRIGHT TREMAINE, LLP, 2600 CENTURY SQUARE, 1501 FOURTH AVENUE, SEATTLE, WA, 98101-1688, US

(21) Appl. No.: 10/502,999

(22) PCT Filed: Jan. 30, 2003
(86) PCT No.: PCT/US03/02951

(30) Foreign Application Priority Data
Jan. 30, 2002 (US) 60352944

(51) Int. Cl. G01N 33/48

(52) U.S. Cl. 436/63

(57) ABSTRACT

The present invention provides a novel method for the systematic identification of differentially methylated CpG dinucleotides positions within genomic DNA sequences for use as reliable markers to detect and characterize different stages of development or differentiation of cells corresponding to different classes of biological samples. Particular embodiments comprise the use of genome-wide discovery techniques for identification of differentially methylated CpG dinucleotide sequences, further identification of neighboring differentially methylated CpG dinucleotide sequences, scoring of the identified differentially methylated CpG positions according to discrimination indices, and confirmation of the predictive utility of selected differentially methylated CpG dinucleotide among a larger set of biological samples. The method, and kits for implementation thereof, are useful in applied assays for distinguishing between different stages of development or differentiation of cells belonging to different classes of biological samples.

### FIELD OF THE INVENTION

The present invention relates to genomic DNA sequences that exhibit altered CpG methylation patterns between and/or among different states of cellular differentiation or development. Particular embodiments provide a systematic method for the efficient identification, assessment and validation of differentially methylated genomic CpG dinucleotide sequences as markers to detect and characterize different stages of cellular development or differentiation.

### BACKGROUND

Significant developments in medical science have arisen over the past decade, reflecting an increased understanding of the human genome. However, even with completion of the sequencing of the Human Genome, fundamental questions remain concerning the mechanisms by which the genome is controlled, and the relationship between such mechanisms and the differentiation process determining a cell's function, and the relationship between such mechanism and cellular differentiation.

Genetic approaches. The vast majority of efforts to identify genomic abnormalities has been, and continues to be based on nucleotide sequence analysis; that is genetic based. During initial phases of the human genome project, genomic markers were linked to disease conditions by mapping. Such mapping techniques involved correlation of the incidence of a disease condition with inheritance of genomic ‘markers’ within a pedigree. Examples of such markers include restriction enzyme sites, visible chromosomal abnormalities such as translocations, single nucleotide polymorphisms and other mutations (e.g., microsatellite DNA, inversions, transversions, deletions, etc.).

Relatively new fields such as proteomics and mRNA analysis (e.g., expression profiling) are also rapidly gaining in importance.

Epigenetic approaches. Additionally, a new and significant epigenetic field relating to DNA methylation pattern analysis is emerging. DNA methylation is the most common covalent modification of genomic DNA. The covalent attachment of a methyl group at the C5-position of the nucleotide base cytosine is particularly common within CpG dinucleotides of gene regulatory regions. The likelihood of finding any particular dinucleotide sequence in a given DNA sequence is 1/16 or ˜6%. In humans, however, the average genomic measured frequency of the CpG dinucleotide is very low (about 1/70). However, contiguous genomic regions of between 300 bp and 3000 bp in length exist, where the occurrence of CpG dinucleotides is significantly higher than normal. These CpG-rich regions are referred to in the art as CpG ‘islands’ and represent about 1% of the genome.

Such CpG islands have primarily been observed in the 5′-region of genes, and more than 60% of human promoters are contained in, or overlap with such CpG islands. Cytosine methylation within such CpG islands plays an important role in gene expression and regulation, in maintenance of normal cellular functions. Moreover, aberrant methylation patterns have been linked with a variety of disease conditions, and in particular with cancer. Many CpG islands are not in the promoters of genes, and their significance and function remains unclear.

Furthermore, cytosine methylation is associated with genomic imprinting and embryonic development (see e.g., Reik & Walter, Nat. Rev. Genet. 2:21-32, 2001; Reik et al., Science 293:1089-1093, 2001). Aberrant imprinting disturbs development and is the cause of various disease syndromes. The study of imprinting also provides new insights into epigenetic gene modification during development.

Deficiencies in the art with respect to assessing cellular differentiation. It is the aim of a number of tissue engineering groups to develop and produce a new tissue or cell lines in a reliable and reproducible manner, and which will eventually gain regulatory approval. For this purpose it is required that: a) cells can be maintained and expanded without changing their phenotype and differentiation status; b) cells can be manipulated and differentiated in a targeted, standardized and efficient way to obtain the desired cell type; and that c) exact lineage, functionality, homogeneity and differentiation status can be assessed.

In early stages of differentiation and growth experiments, the result assessment addresses whether correct progenitor cells were chosen, or whether the subject differentiation pathways are the anticipated ones likely to yield correct tissues. In more advanced stages of product development, the assessment centers on proof of product quality. In this context “correct” is understood as fully biologically functioning with respect to the cell type in question.

Currently, the state of the art in assessing the above-described requirements is based on the analysis of phenotypic changes, such as morphological and biochemical changes of the cells. Typical art-recognized technologies are immuno-histochemical analyses, fluorescent activated cell sorting (FACS), and expression analysis of specific marker proteins. Such biochemical assays often are inconclusive, lengthy, time consuming and not suitable for high-throughput analyses. Additionally, such methods do not always provide for a prediction of the intended cellular function, and are often only meaningful at the end of the differentiation process.

A standard method to determine a cell's state is the use of immuno-histochemical assays. These are based on the detection of specified proteins, mostly surface proteins, and can only address a limited number of proteins of interest. Nonetheless, the more marker proteins are known, the more precisely a cell's differentiation status can be determined using such techniques. However, without the additional use of molecular biology techniques, such as RNA based cDNA/oligo-microarrays or a complex proteomics experiment, which enable the simultaneous view of a larger number of changes, cell differentiation itself and effects of growth factors on differentiation cannot adequately be assessed by such standard techniques as immuno-histochemical assays.

While proteomic approaches have yet to overcome basic difficulties, such as reaching sufficient sensitivity, approaches using RNA-based techniques to analyze expression patterns are well-known and widely used. For example, microarray-based expression analysis studies on cell differentiation is a growing area of research. However, a significant drawback of this technology is its dependency on RNA. Despite extensive research with RNA, the general problem of its instability is not solved. Therefore, each single experiment with RNA needs to take into account that degradation of RNA will occur during the experimental procedure. This problem is aggravated by the fact that RNA expression levels change gradually, so that for the majority of genes the actual expression changes are overlapped and blurred by changes through random degradation. As the variation of concentration of mRNA is high, the experimental procedure required to provide meaningful results from microarray experiments is correspondingly complicated.

Potential advantages of inethylation-based approaches. Significantly, regulatory agencies are currently not willing to accept a technology platform relying on an expression microarray, because of the inherent shortcomings of the method.

In contrast, the technology of methylation analysis is based on the stable DNA molecule, rather than on labile RNA molecules, and depends on a digital-type signal (0/1; caused by a base being either methylated or not). Therefore, results are more sensitive and reliable than for RNA-dependent technologies. A platform based on this technology, if developed would be more likely to be accepted by regulatory authorities.

Specific cell types can be correlated with specific methylation patterns. This has been shown for a number of cases. For example, Adoran et al. describe that it is not only possible to distinguish between healthy tissue and carcinoma tissue, but also to distinguish between tissues derived from different organs (Adoijan et al., Nucleic Acids Res. 30:e21, 2001). DNA modification by cytosine methylation has also been described to occur at specific sites in the genome during the process of in vitro aging (Halle et al., Mutat Res. 316:157-171, 1995).

Furthermore the epigenetic status of toti-potential or pluri-potential stem cells has also been investigated. Pluri-potential stem cells of the mouse are continuously maintained in an undifferentiated state, and are capable of expansion in numbers through rapid cell divisions. Under appropriate conditions, these cells will differentiate into ectodermal, mesodermal and endodermal derivatives in the formation of embryoid bodies following in vitro suspension culture, and in teratoma formation by in vivo transplantation.

These differentiation states will show different methylatibn patterns. Generally, little is known about the epigenetic status of toti-/pluri-potential stem cells, but it has been shown that mouse ES (embryonic stem) cells are hypomethylated in comparison to considerably differentiated somatic cells (see e.g., Tada & Tada, Cell Structure and Function 26:149-160, 2001).

There is also evidence from studies with murine cell lines that specialized cell lineages derived from a common stem cell, and mediated by lineage-specific growth factors, are distinguishable based upon differential methylation status of one or more specific genes (Felgner et al., Leukemia 13:530-534, 1999). Another study shows clearly how the methylation of specific CpG sites in a specific gene (GLUT4) differs due to the cell's differentiation from pre-adipocytes to adipocytes (Yokomori et al., (1999)). These studies have all been performed with cells in cell culture.

Misregulation of genes, leading to other than the expected or desired cell types, may be predicted by comparing the methylation patterns of their progenitor cells with those progenitor cells that develop into the desired cell types.

There is a strong need in the art for additional investigation of the specific location and methylation status of CpG positions within relevant genes to define and enable reliable use of CpG methylation patterns as a marker for cell differentiation states. Such analyses might encompass different cell types and cell states of interest, or include ranges of differentiation.

Methlylation assays. Various methods are currently used in the art for the analysis of specific CpG dinucleotide methylation status. These may be roughly characterized as belonging to one of two general categories: namely, restriction enzyme based technologies, or unmethylated cytosine conversion based technologies.

Restriction enzyme based technologies. The use of methylation sensitive restriction endonucleases for the differentiation between methylated and unmethylated cytosines is perhaps the oldest, and most widely-recognized technique. Restriction enzymes characteristically hydrolyze (cleave) DNA at and/or upon recognition of specific sequences (ie., recognition motifs) that are typically between 4- to 8-bases in length. Among such enzymes, methylation sensitive restriction enzymes are distinguished by the fact that they either cleave, or fail to cleave DNA according to the cytosine methylation state present in the recognition motif (e.g., the CpG sequences thereof).

In methods employing such methylation sensitive restriction enzymes, the digested DNA fragments are typically separated (e.g. by gel electrophoresis) on the basis of size, and the methylation status of the sequence is thereby deduced, based on the presence or absence of particular fragments. Preferably, a post-digest PCR amplification step is added wherein a set of two oligonucleotide primers, one on each side of the methylation sensitive restriction site, is used to amplify the digested DNA. PCR products are not detectable where digestion of the subtended methylation sensitive restriction enzyme site occurs.

The applicability of this technique, in many cases, is limited by the few species of enzymes available and the distribution of their corresponding recognition motifs. Furthermore, these techniques are costly, time consuming, and result in the analysis of only individual sites per reaction. Nonetheless, restriction enzyme based technologies have proven utility for genome-wide assessments of methylation patterns, particularly where sequence data is unavailable. Techniques for restriction enzyme based analysis of genomic methylation include the following: differential methylation hybridization (DMH) (Huang et al., Human Mol. Genet. 8: 459-70, 1999); Not I-based differential methylation hybridization (see e.g., (Kutsenko et al., NAR 30:3163-3170, 2002; WO 02/086163 A1); restriction landmark genomic scanning (RLGS) (Plass et al., Genomics 58:254-62, 1999); methylation sensitive arbitrarily primed PCR (AP-PCR) (Gonzalgo et al., Cancer Res. 57: 594-599, 1997); methylated CpG island amplification (MCA) (Toyota et. al., Cancer Res. 59: 2307-2312, 1999).

Cytosine conversion based technologies. A more common and utilitarian method of CpG methylation status analysis comprises methylation status-dependent chemical modification of CpG sequences within isolated genomic DNA, or within fragments thereof, followed by DNA sequence analysis. Chemical reagents that are able to distinguish between methylated and non methylated CpG dinucleotide sequences include hydrazine, which cleaves the nucleic acid, and the more preferred bisulfite treatment. Bisulfite treatment followed by alkaline hydrolysis specifically converts non-methylated cytosine to uracil, leaving 5-methylcytosine unmodified (Olek A., Nucleic Acids Res. 24:5064-6, 1996). The bisulfite-treated DNA may then be analyzed by conventional molecular biology techniques, such as PCR amplification, sequencing, and detection comprising oligonucleotide hybridization.

Herman and Baylin first described the use of methylation-sensitive primers for the analysis of CpG methylation status with isolated genomic DNA (Herman et al. Proc. Natl. Acad. Sci. USA 93:9821-9826, 1996, and by U.S. Pat. No. 5,786,146; see also U.S. Pat. No. 6,265,171). The described method, methylation sensitive PCR MSP), allows for the detection of a specific methylated CpG position within, for example, the regulatory region of a gene. The DNA of interest is treated such that methylated and non-methylated cytosines are differentially modified (e.g., by bisulfite treatment) in a manner discernable by their hybridization behavior. PCR primers specific to each of the methylated and non-methylated states of the DNA are used in a PCR amplification. Products of the amplification reaction are then detected, allowing for the deduction of the methylation status of the CpG position within the genomic DNA.

Other methods for the analysis of bisulfite treated DNA include inethylation-sensitive single nucleotide primer extension (Ms-SNuPE) (Gonzalgo & Jones, Nucleic Acids Res. 25:2529-2531, 1997; and see U.S. Pat. No. 6,251,594), and the use of real-time PCR based methods, such as the art-recognized fluorescence-based real-time PCR technique MethyLight™ (Eads et al., Cancer Res. 59:2302-2306, 1999; U.S. Pat. No. 6,331,393 to Larid et al.; and see Heid et al., Genoyne Res. 6:986-994, 1996).

However, while the methylation assay methods described herein are useful for the determination of the methylation status of particular genomic CpG positions, and despite continued investigation of the association of diseases with genomic methylation status, the application of methylation status as a reliable marker for cellular differentiation has not emerged.

Presently, there are no commercially available assays for the analysis of the methylation status of CpG dinucleotide sequence positions as markers for cellular differentiation. Significantly, this situation does not reflect any lack of potential for such markers and applications, but rather relates to the fact that there are no known systematic methods for the efficient identification, assessment and validation of such markers.

Therefore, there is a pronounced need in the art for a systematic method for the efficient identification, assessment and validation of differentially methylated genomic CpG dinucleotide sequences as markers for cellular differentiation and development.

### SUMMARY

The present invention provides a method for the identification of differentially methylated CpG dinucleotides within genomic DNA useful as reliable markers to distinguish between different sources of cells.

In particular embodiments, the inventive method comprises four steps. A preferred embodiment comprises a fifth step. A particularly preferred embodiment comprises an additional sixth step (see FIG. 1):

In Step 1, the diagnostic and/or analytical question to be addressed is formulated by identifying at least two different classes of biological samples, characterized as containing genomic DNA. The term ‘identifying’ in this context comprises naming of the relevant samples, and/or sample sets, and preferably sourcing the suitable sets of samples, wherein sourcing the samples means identifying the relevant source and preferably also providing access to those sample sets.

Step 2. Once a suitable set of tissues has been collected, differentially methylated CpG positions are identified within the entire genome, between the two or more classes of samples. The differentially methylated CpG positions are termed ‘Methylated Sequence Tags’ or MeSTs. In a preferred embodiment of the method, Step 2 further comprises a second stage comprising analysis of the literature or other databases to identify CpG positions of interest with respect to the question formulated in Step 1.

In another preferred embodiment, the neighboring sequence context of a differentially methylated CpG position (MeST) is analyzed to further characterize the methylation patterns of the genomic region in question.

In Step 3, these additional CpG positions are scored to select the most promising identified candidate CpG marker positions for further analysis in a fourth step.

In Step 4, CpG positions having utility as reliable “markers” are identified for subsequent analyses. Step 4 consists of two stages. In stage I of Step 4, molecular biological techniques are used to analyze the methylation status of CpG positions identified in the previous steps. This analysis is performed upon a sample set of increased size. Analysis may be carried out by several methods capable of versatile applicability and medium/high throughput (e.g., parallel MS SNuPE). In a particularly preferred embodiment, the analysis is carried out by means of bisulfite treatment, followed by hybridization analysis using an array based format.

In Stage II of the marker identification process, the methylation status of each CpG position is assessed by statistical means as to its suitability for reliable discrimination between said classes of biological samples.

In a preferred embodiment of the method, an additional Step 5 is carried out that comprises ranking of the CpG positions identified according to their capability of distinguishing between said classes of biological samples.

A yet further preferred embodiment comprises design of an applied assay in an additional Step 6, for testing the panel upon a larger sample set.

An alternate embodiment of said method comprises: a) formulating of a cell developmental/cell differentiaion aim of the marker; b) obtaining test and control samples; c) analyzing the samples by means of methods capable of identifying differentially methylated CpG dinucleotide sequences within the entire genome or a representative fraction thereof; d) further investigating the identified CpG positions of interest by analyzing the surrounding sequence context to further characterize the methylation patterns of the genomic region in question; e) further analyzing the identified or surrounding differentially methylated CpG positions within larger sample sets by using a methodology suitable for medium and/or high throughput comparison/screening, wherein the identified or surrounding CpG marker positions are analyzed by statistical means to confirm and identify reliable marker for cellular differentiation.

Preferably, analyzing in c) comprises analysis of the literature and other databases for identification of CpG positions which may be of particular interest with respect to the formulated aim, and optionally comprises relative scoring of the identified CpG positions to facilitate selecting the most promising identified candidate CpG marker positions for farther analysis. Preferably, further investigating in d) comprises a scoring procedure to facilitate selecting a limited subset of the identified markers for further analysis. In a preferred embodiment, the method is implemented in a clinical or laboratory setting.

In alternate embodiments, the present invention provides a method for identification if a reliable marker for development stage or cellular differentiation states characterized by altered DNA methylation, comprising:
a) obtaining a set of at least two biological samples in each case having genomic DNA, wherein the biological samples correspond to at least two sample classes that are distinguishable by a phenotypic or measurable parameter;
b) identifying, using an assay suitable for comparing methylation status between or among corresponding CpG dinucleotide positions within the sample class genomic DNAs, a primary differentially methylated CpG dinucletide sequence position that distinguishes the classes;
c) identifying, within a context DNA region surrounding or including the primary differentially methylated CpG dincleotide position, and using an assay or database suitable therefore, a secondary differentially methylated CpG dinucleotide sequence that distinguishes the classes; and
d) confirming, among a larger set of such biological samples, and using an assay suitable therefore, the class-distinguishing methylation status of the secondary differentially methylated CpG dinucleotide sequence position, whereby a reliable methylation marker is confirmed and provided.

Preferably, identifying a primary differentially methylated CpG dinucleotide sequence in c) comprises analysis of the literature or other databases for identification of CpG positions which may be of particular interest with respect to the formulated aim, and optionally comprises relative scoring of the identified CpG positions to facilitate selecting the most promising primary CpG marker position, or positions, for further analysis. Preferably, identifying a primary or secondary differentially methylated CpG dinucleotide sequence, or a pattern having a plurality of differentially methylated CpG dinucleotide sequences comprises a scoring procedure to facilitate selecting a limited subset of identified primary or secondary differentially methylated CpG dinucleotide sequences, or patterns for further analysis. Preferably, the confirmed class-distinguishing secondary differentially methylated CpG dinucleotide sequence positions identified in d) are ranked according to utility for distinguishing between or among different sample classes.

Preferably, the method is implemented in a clinical or laboratory setting.

### BRIEF DESCRIPTION OF THE DRAWINGS

FIG. 1 shows, in schematic form, components of a method according to the present invention.

### DETAILED DESCRIPTION OF THE INVENTION

The present invention provides, in particular embodiments, a systematic method for the efficient identification, assessment and validation of differentially methylated genomic CpG dinucleotide sequences as markers for cellular differentiation and development.

Definitions:

In this invention “classes of DNA sources” are any distinct sets of samples containing DNA. Preferably said classes are of biological matter. Due to this fact they are referred to herein as ‘classes of biological samples.”

In this context the phrase “phenotypically distinct” shall be used to describe organisms or components thereof, which can be distinguished by one or more characteristics, observable and/or detectable by current technologies. Each of such characteristics may also be defined as a parameter contributing to the definition of the phenotype. Wherein a phenotype is defined by one or more parameters an organism that does not conform to one or more of said parameters shall be defined to be distinct or distinguishable from organisms of said phenotype. Excluded from those characteristics are differences in the organisms' (or the components') cytosine methylation patterns and differences in their DNA sequences.

The term “oligomer” is used whenever a term is needed to describe the alternative use of an oligonucleotide or a PNA-oligomer, which cannot be described as oligonucleotide.

The term “Observed/Expected Ratio” (“O/E Ratio”) refers to the frequency of CpG dinucleotides within a particular DNA sequence, and corresponds to the [number of CpG sites/(number of C bases×number of G bases)]×band length for each fragment.

The term “CpG island” refers to a contiguous region of genomic DNA that satisfies the criteria of (1) having a frequency of CpG dinucleotides corresponding to an “Observed/Expected Ratio”>0.6, and (2) having a “GC Content”>0.5. CpG islands are typically, but not always, between about 0.2 to about 1 kb in length, and may be as large as about 3 Kb in length.

The term “methylation state” or “methylation status” refers to the presence or absence of 5-methylcytosine (“5-mCyt”) at one or a plurality of CpG dinucleotides within a DNA sequence. Methylation states at one or more particular pahndromic CpG methylation sites (each having two CpG CpG dinucleotide sequences) within a DNA sequence include “unmethylated,” “fully-methylated” and “hemi-methylated.”

The term “hemi-methylation” or “hemimethylation” refers to the methylation state of a palindromic CpG methylation site, where only a single cytosine in one of the two CpG dinucleotide sequences of the palindromic CpG methylation site is methylated (e.g., 5′-CCMGG-3′ (top strand): 3′-GGCC-5′ (bottom strand)).

The term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample.

The term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample.

The term “microarray” refers broadly to both “DNA microarrays” and “DNA chip(s),” and encompasses all art-recognized solid supports, and all art-recognized methods for affixing nucleic acid molecules thereto or for synthesis of nucleic acids thereon.

“Genetic parameters” are mutations and polymorphisms of genes and sequences further required for their regulation. To be designated as mutations are, in particular, insertions, deletions, point mutations, inversions and polymorphisms and, particularly preferred, SNPs (single nucleotide polymorphisms).

“Epigenetic parameters” are, in particular, cytosine methylations. Further epigenetic parameters include, for example, the acetylation of histones which, however, cannot be directly analyzed using the described method but which, in turn, correlate with the DNA methylation.

The term “bisulfite reagent” refers to a reagent comprising bisulfite, disulfite, hydrogen sulfite or combinations thereof, useful as disclosed herein to distinguish between methylated and unmethylated CpG dinucleotide sequences.

The term “Methylation assay” refers to any assay for determining the methylation state of one or more CpG dinucleotide sequences within a sequence of DNA.

The term “MS.AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, and described by Gonzalgo et al., Cancer Research 57:594-599, 1997.

The term “MethyLight™” refers to the art-recognized fluorescence-based real-time PCR technique described by Eads et al., Cancer Res. 59:2302-2306, 1999.

The term “HeavyMethyl™” assay, in the embodiment thereof implemented herein, refers to a HeavyMethyl™ MethylLight™ assay, which is a variation of the MethylLight™ assay, wherein the MethylLight™ assay is combined with methylation specific blocking probes covering CpG positions between the amplification primers.

The term “Ms-SNuPE” (Methylation-sensitive Single Nucleotide Primer Extension) refers to the art-recognized assay described by Gonzalgo & Jones, Nucleic Acids Res. 25:2529-2531, 1997.

The term “MSP” (Methylation-specific PCR) refers to the art-recognized methylation assay described by Herman et al. Proc. Natl. Acad. Sci. USA 93:9821-9826, 1996, and by U.S. Pat. No. 5,786,146.

The term “COBRA” (Combined Bisulfite Restriction Analysis) refers to the art-recognized methylation assay described by Xiong & Laird, Nucleic Acids Res. 25:2532-2534, 1997.

The term “MCA” (Methylated CpG Island Amplification) refers to the methylation assay described by Toyota et al., Cancer Res. 59:2307-12, 1999, and in WO 00/26401A1.

The term “hybridization” is to be understood as the binder of a bond of an oligonucleotide to a complementary sequence along the lines of the Watson-Crick base pairings in the sample DNA, forming a duplex structure.

“Stringent hybridization conditions,” as defined herein, involve hybridizing at 68° C. in 5×SSC/5× Denhardt's solution/1.0% SDS, and washing in 0.2×SSC/0.1% SDS at room temperature, or involve the art-recognized equivalent thereof (e.g., conditions in which a hybridization is carried out at 60° C. in 2.5×SSC buffer, followed by several washing steps at 37° C. in a low buffer concentration, and remains stable). Moderately stringent conditions, as defined herein, involve including washing in 3×SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

The phrase “sequence context of selected CpG dinucleotide sequences” refers to a genomic region of from 2 nucleotide bases to about 3 Kb surrounding or including a primary differentially methylated CpG dinucleotide identified by the genome-wide Discovery methods described herein (in Step 2 of the inventive method). Said context region comprises, according to the present invention, at least one secondary differentially methylated CpG dinucleotide sequence, or comprises a pattern having a plurality of differentially methylated CpG dinucleotide sequences including the primary and at least one secondary differentially methylated CpG dinucleotide sequences. Preferably, the primary and secondary differentially methylated CpG dinucleotide sequences within such context region are comethylated in that they share the same methylation status in the genomic DNA of a given tissue sample. Preferably the primary and secondary CpG dinucleotide sequences are comethylated as part of a larger comethylated pattern of differentially methylated CpG dinucleotide sequences in the genomic DNA context. The size of such context regions varies, but will generally reflect the size of CpG islands as defined above, or the size of a gene promoter region, including the first one or two exons.

Unless defined otherwise, all technical and scientific terms used herein have the same meaning as commonly understood by one of ordinary skill in the art to which the invention pertains. Although any methods and materials similar or equivalent to those described herein can be used for testing of the present invention, the preferred materials and methods are described herein. All documents cited herein are thereby incorporated by reference.

A Systematic Method for the Efficient Identification of Reliable Differentially Methylated Cellular Differentiation Markers within Genomic DNA

The subject matter of the invention is directed to a method for the identification of informatively methylated CpG dinucleotides within genomic DNA. These may be used either alone or as components of a gene panel cellular differentiation or analytical assay.

In particular, the method according to the invention is directed to the identification of differentially methylated CpG positions which may be used as markers for the classification of cells according to their differentiation developmental states. The invention provides a method to distinguish between cells with distinct phenotypes or other measurable distinguishing parameters in an easier and faster way than it is possible today.

Moreover it provides a method to distinguish between cells, which cannot currently be distinguished by available techniques; that is, they are neither genotypically nor phenotypically distinct, according to the definition given previously. For example, a laboratory cell line, having been kept under laboratory-specific variations of standard culturing conditions, might developed different methylation patterns, without showing any phenotypic differences as assessed by current methods.

To date, there exist no commercially available assays for the analysis of CpG positions as markers for specific differentiation states of cells or for the identification of a specific type of cell and its functionality. Furthermore there are no known systematic methods for the identification, assessment and validation of these markers. The method according to the disclosed invention provides a systematic means for the identification and verification of multiple development-specific and differentiation state relevant CpG positions to be used alone, or in combination with other CpG positions (as a panel of markers), to form the basis of a relevant and reliable analytical assay.

The method according to the invention enables the differentiation between two or more phenotypically or otherwise distinct classes of DNA sources. In most cases these might be classes of biological matter. Due to this fact, throughout the invention it is referred to ‘classes of biological samples’. Said method comprises the comparative analysis of the methylation patterns of CpG dinucleotides within each of said classes. Said method is comprised of four steps. These are outlined in brief here:

Step 1: Identification of at least two different classes of genomic DNA-containing biological samples, to be analyzed in the subsequent steps.

Step 2: Determination of differences in CpG methylation patterns (of the genomic DNA) between said at least two classes of biological samples by means of analysis of the genome-wide methylation patterns of biological samples of both classes. To accomplish this, the methylation status of the CpG positions within each of said samples and/or classes is determined, the results (the methylation status of the analyzed CpG position(s)) between each of said classes are compared, and those CpG positions differentially methylated between said classes are identified.

In a preferred embodiment, an optional stage is added comprising the determination of the characteristic methylation patterns of CpG positions in the vicinity of the differentially methylated CpG positions identified previously, and thereby determining further CpG positions differentially methylated between said classes.

Step 3: Scoring of the CpG positions found to be differentially methylated between said at least two classes of biological samples according to their likelihood or utility for discrimination between said at least two classes of biological samples, the purpose being to select the most promising candidate CpG marker positions identified for further analysis in a Step 4.

Step 4: Identification of the methylation status of said differentially methylated CpG positions identified in Step 2 and scored in Step 3 within larger numbers of samples of each class, and analysis of the data generated to identify CpG positions, which have utility for reliably distinguishing between said classes of biological samples either singularly or in combination with other informative CpG positions.

The method will be described in more detail herein below:

Step 1—Formulating the Problem, Defining the Experimental Design and Sample Collection:

In the first step (Step 1) of the method, the question to be addressed is formulated. The method as described herein may be used to compare two or more types of phenotypically or otherwise distinct classes of biological samples. Said biological sample is characterized as containing DNA. Said sample can be, for example, a cell, a cell compartment or a tissue sample. However said term of ‘biological sample’ is also understood as including nucleic acids or genomes. CpG methylation analysis can for example be used to distinguish between cells, tissues or organisms, which are genotypically identical or similar at the relevant genes. This is independent of whether the cells etc. are phenotypically distinct or not.

In the method according to the present invention, the analytical problem to be addressed is formulated such that two or more phenotypically or otherwise distinct classes of biological matter (hereinafter also referred to as ‘classes’ or ‘classes of biological samples’) are differentiated or distinguished from one another.

For example, in one embodiment of the method the first step is to decide that the analytical problem to solve is to distinguish between fully differentiated chondrocytes and their precursor cells. For these two classes of biological samples the relevant sources are then identified. In another embodiment the two classes of interest are in vitro dedifferentiated chondrocytes and in vivo dedifferentiated chondrocytes.

The question to be formulated should be relevant with regards to an existing problem, such as the differentiation of glucose-responsive in vitro developed B-cells and non-glucose-responsive in vitro developed B-cells. It should be technically feasible and preferable to have a significant commercial market size for an analytical assay. For example the system as described herein may be used for the development of analytical tools for the grading and staging of cultured cells used for tailored differentiation purposes, for use in pre-surgery quality assessment of tissues or cells to be implanted, and for the post-surgery evaluation of the implanted material.

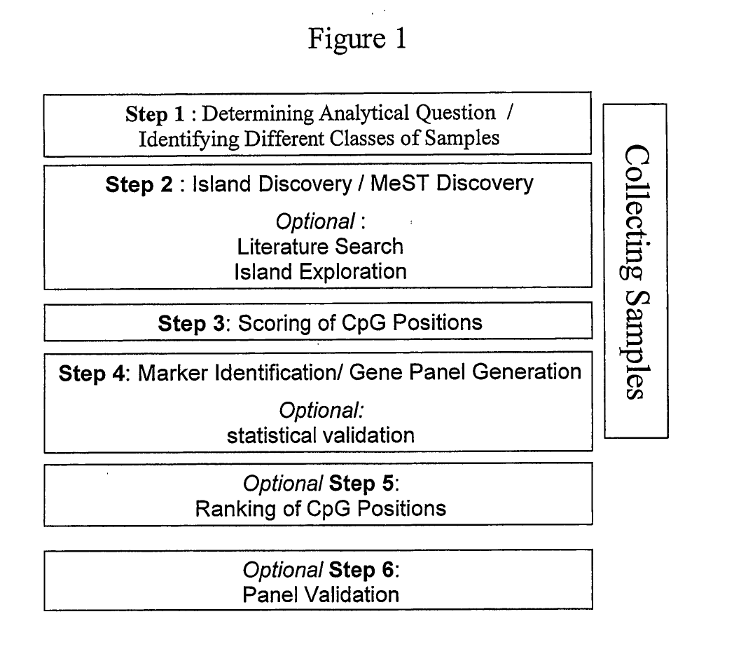

In a preferred embodiment of the method, suitable biological samples are sourced and acquired subsequent to the formulation of the diagnostic aim of the marker. Sourcing and acquisition of the samples may be completed prior to the initiation of the next step (Step 2) or in a preferred embodiment of the method sourcing and acquisition of the samples may be ongoing with the following steps of the method (see FIG. 1).

Samples may be obtained according to standard techniques from all types of biological sources that are usual sources of DNA such as, but not limited to, cell lines, cells or cellular components which contain DNA, biopsy samples, autopsy samples, bodily fluids such as, but not limited to, blood, sputum, stool, urine, ejaculate, or cerebrospinal fluid, and also tissue embedded in paraffin such as but not limited to, tissue from eyes, intestine, kidney, brain, heart, prostate, lung, breast, liver, histological object slides, and all possible combinations thereof.

Samples should be representative of the target population and should be as unbiased as possible. It is a preferred embodiment that the first step includes planning and organizing how to provide the samples required not only in Step 2, but also for the subsequent steps. Preferably, during Step 2 of the method the genomic DNA should be obtained from a high quality source (e.g., said sample should contain only the tissue type of interest, and minimum contamination and minimum DNA fragmentation). Preferably, during Step 2, each class to be analyzed should be represented by a sample set size of 10 or above. However, during Step 4 samples should be representative of the type that is to be handled by the applied diagnostic assay, (i.e., may be of less pure quality and samples are analyzed individually rather than pooled). For Step 4, analysis is carried out on sample set sizes in the hundreds.

In all the subsequent steps of said method, methylation levels of CpG positions are compared between said at least two classes, to identify CpG positions differentially methylated between said classes. To minimize the variables between the at least two classes, each class may be further segregated into sets according to predefined parameters.

Once suitable sets of tissue samples have been established (number of samples being preferably 10 or above, all of high quality, and in a preferred embodiment the sample set consists of tester and driver matched pair samples for comparison), Step 2 of the method may be initiated. This step is herein also referred to as ‘CpG Island Discovery’ or simply ‘Island Discovery.’

Step 2—CpG Island Discovery:

The aim of this step of the method is to survey the entire genome or a representative portion thereof for phenotypically or otherwise characteristic CpG methylation patterns. CpG positions representative of a significant proportion of the genome are analyzed to ascertain the methylation status of the different classes on a genome-wide basis or level. The methylation pattern of each sample set is characterized and CpG positions differentially methylated between the sets are identified. In a preferred embodiment, at least 50 different CpG positions are analyzed, and in a particularly preferred embodiment the analyzed CpG positions are situated within at least 20 different discrete genes and or their promoters, introns, first exons and/or enhancers.

Step 2 is comprised of two stages, Stage II being optional. Both stages identify CpG positions, which may be of interest with respect to the question formulated in Step 1. The CpG positions which are identified as being differentially methylated between the sample sets in this step of the method are termed ‘Methylated Sequence Tags’ or MeSTs. In Stage I this is done by employing molecular biological methods while Stage II utilizes the published state of the art to identify further CpG positions of interest.

Stage I of Step 2 (MeST—Discovery). In Stage I the methylation pattern of each sample set is characterized, and CpG positions differentially methylated between the sets are identified.

Preferably, the methods used to characterize the methylation patterns of each sample set (hereinafter also referred to as ‘Discovery techniques’) enable a genome-wide methylation pattern analysis. In a particularly preferred embodiment, the characterization is carried out by means of methylation sensitive restriction enzyme digest analysis, and in particular by means of one or a combination of the following techniques: Methylated CpG island amplification (MCA); Arbitrarily primed PCR (AP-PCR); Restriction landmark genomic scanning (RLGS); Differential methylation hybridization (DMH, also known as ECIST); and NotI restriction based differential hybridization method.

A more detailed explanation of some of the preferred discovery techniques follows:

Differential methylation hybridization (DMH). DMH is a microarray compatible approach that simultaneously detects DNA methylation in thousands of CpG islands. The first part of DMH is the generation of multiple CpG island tags (CGI library) as templates arrayed onto solid supports (e.g., glass slides or nylon membranes). The generation of CpG island tags has been described (Huang et al., Human Mol. Genet. 8, 459-70, 1999). Briefly, genomic DNA is isolated, purified and digested using a restriction enzyme that is unlikely to digest within CpG islands, for example MseI (TTAA). The DNA digest is then enriched for CpG-rich regions (e.g., by in vitro methylation of the digest and purification using a methylated DNA binding column consisting of a polypeptide of the DNA binding domain of the rat MeCP2 protein attached to a solid support; as described by Cross et. al. Nature Genetics 6:236-244, 1994). The restriction fragments are screened for repeat elements and PCR amplified. The fragments are then fixed in the form of an array on a solid surface (e.g., glass slide, nylon membrane), in a manner whereby each fragment is locatable and identifiable on the surface.

The second part involves preparation of amplicons, corresponding to test and reference (control) genomes. Amplicons are used as probes in array-hybridization. Breifly, for amplicon generation, genomic DNA from both the test and reference samples are isolated. Each DNA sample is digested using an enzyme unlikely to digest within CpG islands (e.g., the same enzyme as was used to generate the CGI library). Linker sequences are ligated to the ends of the DNA fragments, and the DNA fragments digested using one or more methylation sensitive restriction enzymes. The digest fragments are PCR amplified and labeled. No PCR amplificate is detectable where the restriction of a fragment has taken place during the second digest. The labeled PCR products are hybridized to the CGI library generated earlier. Comparison of the hybridization pattern of PCR fragments from different types of tissues allows for the detection of differences in methylafion patterns between the two types of tissues. Positive signals identified by the test amplicon, but not by the reference amplicon, indicate the presence of hypermethylated CpG island loci in test cells.

Restriction landmark genomic scanning (RLGS). In RLGS-based methods, differential methylation of CpG positions is discriminated based on digestion of genomic DNA with a methylation sensitive restriction endonuclease. RLGS provides quantitative analysis of CpG islands separated by two-dimensional gel electrophoresis into discrete spots. The resulting spot patterns, or RLGS profiles, are highly reproducible, and thus amenable to intra- and inter-individual comparison.

In a particularly preferred embodiment, each sample is analyzed as a member of a paired set for comparison. DNA is extracted using standard methods known in the art (e.g., by using commercially available kits). Each sample is treated to prevent random labeling of the DNA strands. The treated DNA is digested using a landmark restriction enzyme, for example but not limited to, NotI. The restriction enzyme is deactivated and the digest fragments are labeled at the restriction site. Cleaved landmark restriction sites are preferably labeled with a radioisotope. The genomic DNA is further fragmented, in a progressive manner, with restriction endonucleases with sequence recognition specificity that does not recognize sequences containing CpG, to separate the CpG islands.

For two purposes of dimensional separations, the digest fragments are separated by size, for example by using a high-resolution gel electrophoresis in a first dimension. The nucleic acid fragments are subjected to a restriction enzyme digest carried out in the gel. After digestion, the fragments are electrophoresed a second time with the current running perpendicular relative to the direction of the current in the first electrophoresis. Each gel is exposed using X-ray film or other such suitable methods compatible with the detectable label used to produce a fixed image of the positions of the fragment within the gel. The highly reproducible DNA fragment patterns on the x-ray films exposed to each of the 2-dimensional gels (referred to as “RLGS Profiles”) are then compared to determine where the patterns differ.

Methylation-Sensitive Arbitrarily-Primed Polymerase Chain Reaction (NS.AP-PCR). MS.AP-PCR refers to the art-recognized technology that allows for a global scan of the genome using CG-rich primers to focus on the regions most likely to contain CpG dinucleotides, and described by Gonzalgo et al., Cancer Research 57:594-599, 1997. For present inventive applications of MS.AP-PCR methods, the two classes of DNA samples are each digested with at least one species of restriction endonuclease, of which at least one is a methylation sensitive restriction endonuclease. The digested fragments are amplified in a PCR reaction of variable stringency, as determined by the investigator. At least one of the primers used in the amplification reaction is/are arbitrarily designed. PCR amplificates from both test and driver samples are compared to identify CpG positions differentially methylated between the test and driver classes.

Methylated CpG island amplification (MCA). MCA is based on sequential restriction enzyme digestion with methylation-sensitive/insensitive isoschizomers, adaptor ligation and whole-methylated-genome PCR. A first digestion is carried out upon the genomic DNA of interest using a methylation sensitive restriction enzyme (e.g., SmaI). SmaI is a methylation sensitive restriction enzyme that does not cut when its recognition sequence CCCGGG contains a methylated CpG position, whereas unmethylated CpG positions are digested leaving blunt edged fragments. The SmaI digest is redigested using the methylation insensitive isoschizomer of the enzyme used previously, said digestion leaving sticky ends. For example, SmaI digests are digested by use of the SmaI isoschizomer XmaI, which leaves a sticky edged CCGG overhang. Adaptors are then ligated to the sticky ends and the fragments are amplified, preferably by means of PCR. The amplificate fragments may then be analyzed using a number of methods (e.g., chromatographic methods, sequencing, hybridization analysis) for analysis and comparison of methylation status both within and between classes of tissue. In a preferred embodiment of the method, said analysis is carried out by hybridization of the test to the driver amplificates and subtraction of the fragments common to both.

Stage II of Step 2 (Literature Search). In a preferred embodiment of the method Stage I of Step 2 is supplemented by a second stage. In this Stage II a literature search is conducted including genome databases and peer reviewed publications of the art in order to identify CpG positions which may be of interest with respect to the question formulated in Step 1, and which may be used to distinguish between said classes of samples.

In a particularly preferred embodiment of the inventive method, the candidate marker CpG positions are further assessed by using a scoring system to rank MeSTs according to their potential as marker candidates for progression to Step 3 of the method.

Thus, step 2 provides for a method for identifying one or more primary differentially methylated CpG dinucleotide sequences of a test subject genomic DNA using a controlled assay suitable for identifying at least one differentially methylated CpG dinucleotide sequences within the entire genome, or a representative fraction thereof.

The two groups of CpG positions thus identified in Stages I and II, are combined. The techniques that are used in Stages I and II (of Step 2 of the method) allow for the identification of CpG positions of interest, however they do not provide detailed information about the methylation patterns of the sequence context in which they occur. In a preferred embodiment Step 2 consists of a third stage,

Stage III of Step 2 (Island Exploration). The techniques used above allow for the identification of particular CpG positions of interest without providing information about the methylation patterns of the sequence context in which they occur. In stage III of Step 2 of the method, the sequence context of the MeSTs are investigated to ascertain methylation patterns of one or more surrounding CpG dinucleotide sequences. CpG positions occurring in CpG-rich islands of the genome are often co-methylated (wherein a significant proportion of the CpG positions within the island share the same methylation status). It is particularly preferred that marker positions occur in co-methylated islands to enable easier assay development.

The phrase “sequence context of selected CpG dinucleotide sequences” refers, for purposes of the present invention, to a genomic region of from 2 nucleotide bases to about 3 Kb surrounding or including a primary differentially methylated CpG dinucleotide identified by the genome-wide Discovery methods described herein (in Step 2 of the inventive method). Said context region comprises, according to the present invention, at least one secondary differentially methylated CpG dinucleotide sequence, or comprises a pattern having a plurality of differentially methylated CpG dinucleotide sequences including the primary and at least one secondary differentially methylated CpG dinucleotide sequences. Preferably, the primary and secondary differentially methylated CpG dinucleotide sequences within such context region are comethylated in that they share the same methylation status in the genomic DNA of a given tissue sample. Preferably the primary and secondary CpG dinucleotide sequences are comethylated as part of a larger comethylated pattern of differentially methylated CpG dinucleotide sequences in the genomic DNA context. The size of such context regions varies, but will generally reflect the size of CpG islands as defined above, or the size of a gene promoter region, including the first one or two exons.

Analysis of the sequence context of the MeSTs is generally taken, in the case of inventive gene associated CpG sequences, to be sequence analysis of the promoter and first exon regions of associated genes, and/or the CpG island within which the MeST lies, but this is left to the discretion of a person skilled in the art.

Said analysis may be carried out by any means known in the art (e.g., restriction enzyme based technologies, probe hybridization etc.), however, in the most preferred embodiment of the method said step is carried out by means of bisulfite treatment of the genomic DNA followed by sequencing.

The procedure that is described here is based on the bisulfite-dependent modification of all non-methylated cytosines to uracil, which exhibits the same base pairing behavior as thymine. Sodium bisulfite reacts with the 5, 6-double bond of cytosine, but not with methylated cytosine. Cytosine reacts with the bisulfite ion to form a sulfonated cytosine reaction intermediate, which is susceptible to deamination, giving rise to a sulfonated uracil. The sulfonate group can be removed under alkaline conditions, resulting in the formation of uracil. Uracil is recognized as a thymine by polymerase and thereby upon PCR, the resultant product contains cytosine only at the position where 5-methylcytosine occurs in the starting template DNA. Thus, in DNA treated with bisulfite, 5-methylcytosine can easily be detected by virtue of its hybridization to guanine. This enables the use of variations of established methods of molecular biology, such as sequencing. Sequencing of bisulfite-treated DNA has been described (see e.g., Grunau C, et al., Nucleic Acids Res. 29:E65-5, 2001).

Sequencing of the bisulfite-treated DNA may be carried out using any technique standard in the art, such as the Maxam-Gilbert method and other methods such as sequencing by hybridization (SBH), but is most preferably carried out using the Sanger method. Primer selection is crucial in bisulfite based methylation analysis, since the complexity of DNA is reduced (unless methylation is present, there are only 3 bases on the strand). It is preferred that said primers be designed such that they do not contain any CpG dinucleotide. Furthermore, in a preferred embodiment of the method, they are analyzed for specificity by testing them on genomic DNA (where no amplificates should be obtained).

A further preferred embodiment employs the cycle-sequencing method, also called linear amplification sequencing (see e.g., Stump et al., Nucleic Acids Res., 27:4642-8, 1999; Fulton & Wilson Biotechniques 17:298-301, 1994). Like the standard PCR reaction, it uses a thermostable DNA polymerase and a temperature cycling format of denaturation, annealing and DNA synthesis. The difference is that cycle sequencing employs only one primer and includes a ddNTP chain terminator in the reaction. The use of only a single primer means that unlike the exponential increase in product during standard PCR reactions, the product accumulates in a linear manner. Because the product accumulates during the reaction, and because of the high temperature at which the sequencing reactions are carried out, and the multiple heat denaturation stages, small amounts of double stranded plasmids, cosmids and PCR products may be sequenced reliably without a separate heat denaturation step.

In a further embodiment of the inventive method, samples of DNA are pooled with other members of their class thereby requiring only one sequencing reaction per class. Subsequent to sequencing it may be apparent that both methylated and unmethylated versions of each CpG position are detected within a class thereby allowing an assessment of the degree of methylation of a CpG position within a specific class.

In a preferred embodiment of the method, unsuitable candidate marker CpG positions may be eliminated by means of a scoring system (as carried out in Step 2) subsequent to sequencing of bisulfite-treated DNA. It is particularly preferred that CpG positions not exhibiting co-methylation (methylation of multiple CpG positions) within the examined ‘contex’ region are not analyzed in the subsequent steps of the inventive method.

Thus, stage III of Step 2 provides for identifying, within a genomic DNA context region surrounding or including one or more primary differentially methylated CpG dincleotides, and using an assay suitable therefore, one or more secondary differentially methylated CpG dinucleotide sequences, or a pattern having a plurality of differentially methylated CpG dinucleotide sequences and including the primary and at least one secondary differentially methylated CpG dinucleotide sequences.

Step 3—Scoring:

Investigation of all identified candidate CpG positions is likely to be unproductive and costly. Unsuitable candidate marker CpG positions may be eliminated by means of the scoring system subsequent to bisulfite sequencing. Therefore in Step 3 of the method, subsequent to Step 2, each candidate CpG position is scored as to its suitability for further analysis. The scoring comprises of assessing at least one or a combination of several of the following parameters:
1. Confirmation of the MeST. The more techniques have been used to confirm the same result the better. In cases where it has been possible to identify the MeST using only one technique, that MeST is scored low. However, where it has been possible to verify its variable methylation status using multiple techniques, it gets a higher scoring.
2. Tissue specificity. Whenever the same MeST shows up in different classes of DNA sources, its scoring needs to be reduced as its ability as a specific marker is lowered. However, it needs to be considered whether this was achieved using the one method or multiple methods.
3. Sequence context. MeSTs that contain CpG positions occurring in an area that may be of further interest e.g. within a CpG island or close to a gene that has been already identified as a marker score higher than MeSTs that contain CpG positions occurring within microsatellite DNA.
4. Gene association. If the MeST is associated with a gene, it is important where it is located, e.g. promoter region, coding region, intron or 3′-region. MeSTs within the 5′-promoter region are the most suitable candidates for further investigation, they will get a high score. If the MeST is associated with a gene, it gets an even higher score, if it is a gene of interest. For example, if the DNA source was a β-cell, genes that are associated with insulin production would score highly.
5. Association with an implicated gene; that is, if the MeST is associated with a gene, does the associated gene have known functional or etiological relevance (e.g., if the associated gene is implicated in cellular differentiation, the associated MeST would score highly).

It is particularly preferred that CpG positions not exhibiting co-methylation (methylation of multiple CpG positions) are not analyzed in the subsequent steps of the method.

Step 4—Marker Identification:

Step 4, also referred to as the Marker Identification Step, is carried out subsequent to sequencing of bisulfite-treated DNA and scoring. As many samples as possible from all classes of tissue analyzed during Steps 2 and 3, as well as any further classes of tissues that may wish to be compared should be analyzed in Step 4. The total number of samples should ideally be in the hundreds. Typically around 500 individual CpG positions may be investigated with an aim of reducing these to the 5-25 best markers for use singly or in the form of a panel. Step 4 is carried out in two stages.

In Stage I, molecular biological techniques are used to analyze the methylation status of CpG positions identified in the previous steps (2 and 3). The methylation analysis is performed upon a sample set of increased size relative to that prior Steps 2 and 3. Such analysis may be carried out by several methods having versatility and medium/high throughput (e.g., parallel MS-SNuPE). In a particularly preferred embodiment, however, the analysis is carried out by means of bisulfite-treatment followed by oliogonucleotide hybridization analysis using an array-based format.

Stage II of the Marker Identification Step is based on statistical and in silico analysis. In Stage II, the methylation status of each CpG position is assessed by statistical means as to its capability of discriminating between the DNA of the sample classes. CpG positions, which show significant methylation status differences between the classes are then combined to form a panel. Once the panel is defined, algorithmic methods for the classification of a sample, based on the methylation status of the panel CpG positions is developed. A suitable assay is thus developed in order to test the panel upon a larger sample set.

The two stages are explained in more detail herein below:

Stage I of Step 4. In a preferred embodiment of the method stage I of said Step 4 is carried out by means of hybridization analysis. In the most preferred embodiment, said analysis is carried out by means of the following steps:

In the first step of stage 1, the genomic DNA sample is isolated from tissue or cellular sources. Such sources include, but are not limited to, cell lines, histological slides, bodily fluids or tissues embedded in paraffin. Extraction is by means that are standard to one skilled in the art, and include, but not limited to the use of detergent lysates, sonification, vortexing with glass beads, and precipitating with ethanol. Once the nucleic acids have been extracted and preferably purified, the genomic double-stranded DNA is used in the analysis.

In a preferred embodiment, the DNA may be cleaved prior to chemical treatment (below), by an art-recognized method, in particular with restriction endonucleases.

Subsequently, the genomic DNA sample is chemically treated in such a manner that cytosine bases, which are unmethylated at the C5-position are converted to uracil, thymine, or another base, which is detectably dissimilar to cytosine in terms of hybridization properties. This will be referred to hereinafter as ‘pretreatment,’ or, in particular embodiments, ‘bisulfite treatment.’

The above-described treatment of genomic DNA is preferably carried out with bisulfite (sulfite, disulfite) and subsequent alkaline hydrolysis, which results in conversion of non-methylated cytosine nucleobases to uracil, which is detectably dissimilar to cytosine in terms of base-pairing properties.

Fragments of the pretreated DNA are amplified, using sets of primer oligonucleotides and a polymerase. Preferably, the polymerase is a heat-stable polymerase. Preferably, because of statistical and practical considerations, more than ten different fragments having a length of 100-2000 base pairs are amplified. The amplification of several DNA segments can be carried out simultaneously in one and the same reaction vessel. Usually, the amplification is carried out by means of a polymerase chain reaction (PCR).

In a preferred embodiment of the method, the set of primer oligonucleotides includes at least two oligonucleotides (a forward primer and a reverse primer) in each case identical to a sequence comprising about 18 contiguous nucleotides, or more, of the pretreated nucleic acid.

In a particularly preferred embodiment, said set of primer oligonucleotides includes at least one pair of oligonucleotides, wherein said pair includes one oligonucleotide primer which is reverse complementary to a segment of the pretreated sequence to be amplified, and another which is identical to another segment of the pretreated sequence to be amplified. In a particularly preferred embodiment, said segment is at least 18 bases long. Preferably, the primer oligonucleotides do not comprise any CpG dinucleotides.

In a preferred embodiment of the present invention, at least one primer oligonucleotide is bound to a solid phase during amplification. The different oligonucleotide and/or PNA-oligomer sequences can be arranged on a plane solid phase in the form of a rectangular or hexagonal lattice. Preferably, the solid phase surface is composed of silicon, glass, polystyrene, aluminum, steel, iron, copper, nickel, silver, or gold. Other materials, such as nitrocellulose or plastics also have utility as solid phases.

The fragments obtained by means of the amplification (also referred to herein as ‘amplificates’) can carry a directly or indirectly detectable label. Preferred are labels in the form of fluorescence labels, radionuclides, or detachable molecule fragments having a typical mass, which can be detected in a mass spectrometer. Preferably, detachable molecule fragments have a single-positive or single-negative net charge for better detectability in the mass spectrometer. Preferably, the mass spectrometry detection is carried out and visualized using matrix assisted laser desorption/ionization mass spectrometry (MALDI), or using electron spray mass spectrometry (ESI).

The amplificates obtained are subsequently hybridized to an array or a set of oligonucleotides and/or PNA probes.

Preferably, where the amplificate nucleic acid is in solution, hybridization of the amplificates to the detection oligonucleotides or PNA oligomers is conducted in a hybridization chamber at a hybridization temperature that is dependant upon the selection of oligos. Optimal incubation temperatures and times will differ, depending on the particular oligonucleotides or PNA oligomers selected, and appropriate adjustments to the experimental setup can be readily determined by a person skilled in the art. Preferably, hybridization is carried out under moderately stringent to stringent conditions as defined herein above, or the art-recognized equivalent thereof. In a preferred embodiment, the hybridization is conducted at a temperature that is about 0.5° C. to 3° C. lower than the lowest melting temperature of the selected oligonucleotides, for 16 hours in an appropriate buffer solution. In a particular preferred embodiment, the buffer solution contains SSC and sodium laurel sarcosinate and the hybridizing temperature is 42° C. In a further embodiment the hybridization is conducted at a temperature of 45° C. for four hours. Preferably, the hybridization is carried out in Unihybridization solution (1:4 dilution v/v; Telechem).

Preferably, the set of probes used during the hybridization is comprises at least 10 oligonucleotides or PNA-oligomers. In the inventive method, the amplificates serve as probes which hybridize to oligonucleotides previously bonded to a solid phase. The non-hybridized fragments are subsequently removed.

Preferably, said oligonucleotides comprise at least one base sequence having a length of about 13 nucleotides, which is reverse complementary or identical to a segment of the amplificates sequences, wherein the segment comprises at least one CpG, TpG or CpA dinucleotide sequence. In a particularly preferred embodiment, said dinucleotide is located within the middle third of the oligonucleotide. The cytosine of the CpG dinucleotide is the 5^{th} to 9^{th} nucleotide from the 5′-end of the about 13-mer. Preferably, one oligonucleotide exists for each CpG dinucleotide of interest. More preferably, each CpG dinucleotide of interest is analyzed using two oligonucleotides, one comprising a CpG dinucleotide at the position in question and another comprising a TpG dinucleotide at the position in question.

More preferably, said oligonucleotides comprise at least one base sequence having a length of about 18 nucleotides, which is reverse complementary or identical to a segment of the amplificates sequences. Preferably the CpG dinucleotide is located between the 7^{th} and the 11^{th} nucleotide of said segment. Preferably, at least one CpG is located in the middle of said segment. Preferably, not more than two CpG dinucleotides are located in said segment.

Said oligonucleotides may also be in the form of peptide nucleic acids (PNA) comprising at least one base sequence having a length of about 9 bases which is reverse complementary or identical to a segment of the amplificates sequences, wherein the segment comprises at least one CpG dinucleotide. The cytosine of the CpG dinucleotide is the 4^{th} to 6^{th} nucleotide seen from the 5′-end of the about 9-mer. Preferably, one PNA oligomer exists for each CpG dinucleotide. More preferably, each CpG dinucleotide is analyzed by means of two PNA oligonucleotides, one comprising a CpG dinucleotide at the position in question and another comprising a TpG dinucleotide at the position in question.

Therefore, in a particularly preferred embodiments, two oligomers exist for each CpG position, one comprising a CpG dinucleotide at the dinucleotide position to be analyzed, and the other comprising a TpG oligonucleotide at said position (i.e., one oligonucleotide specific for detection of methylated nucleic acids and the other specific for the detection of unmethylated versions of the same nucleic acid). The use of the two species of oligonucleotide on the solid phase enables an analysis of the degree of methylation within a genomic DNA sample. Comparison of the relative amount of nucleic acid hybridized to each species of oligonucleotide enables the deduction of the degree of methylation at the position in question.

In the final step of stage 1 of Step 4 of the method, the hybridized amplificates are detected. Preferably, labels attached to the amplificates are identifiable at each position of the solid phase at which an oligonucleotide sequence is located.

Preferably, the labels of the amplificates include, but are not limited to fluorescence labels, radionuclides, or detachable molecule fragments having a typical mass which can be detected in a mass spectrometer. Preferably, detection of the amplificates, detachable fragments of the amplificates or of probes which are complementary to the amplificates using mass spectrometry is by matrix assisted laser desorption/ionization mass spectrometry (MALDI) (e.g., Karas &Hillenkamp, Anal Chem., 60:2299-301, 1988), or using electron spray mass spectrometry (ESI). Preferably, the produced detachable mass fragments may have a single-positive or single-negative net charge for better detectability in the mass spectrometer.

Preferably, the array of different oligonucleotide- and/or PNA-oligomer sequences is arranged on the solid phase in the form of a rectangular or hexagonal lattice. The solid phase surface is preferably composed of silicon, glass, polystyrene, aluminum, steel, iron, copper, nickel, silver, or gold. However, nitrocellulose as well as plastics such as nylon which can exist in the form of pellets or also as resin matrices are possible as well.

Methods for manufacturing such arrays are well-known in the art, for example, from U.S. Pat. No. 5,744,3051 using solid-phase chemistry and photolabile protecting groups. An overview of the Prior Art in oligomer array manufacturing can be gathered from a special edition of Nature Genetics (Nature Genetics Supplement, Volume 21, January 1999, and from the literature cited therein.

Stage II of Step 4. The analysis of the methylation status of specific CpG positions within a number of samples generates a large amount of data. Sophisticated statistical and data-analysis techniques are applied to organize and analyze the data; that is, to correlate the methylation pattern with the phenotypic characteristics of the examined samples. Statistical analysis employing, for example, a T-test or a Wilcoxon test, can be used to determine the probability (‘p-value’) that the observed distribution of samples between the classes for each specific CpG position occurred by chance. Each CpG position is then ranked according to the p-values observed. Only the CpG positions of the appropriate p-value are used in the panel.

Once the panel is defined, algorithmic methods for the classification of a sample based on the methylation status of the CpG positions within the panel are developed. Preferably, the correlation of the methylation status of the marker CpG positions with the phenotypic parameters is done substantially without human intervention. Machine learning algorithms automatically analyze experimental data, discover systematic structure in it, and distinguish relevant parameters from uninformative ones.

Machine leaning predictors are trained on the methylation patterns (CpG/TpG ratios) at the investigated CpG sites of the samples with known phenotypical or non-phenotype-based classification. The CpG positions which prove to be discriminative for the machine learning predictor are used in the panel. In a particularly preferred embodiment of the method, both methods are combined; that is, the machine learning classifier is trained only on the CpG positions that are significantly differentially methylated according to the statistical analysis. This method is successful in cancer classification (Model, F., Adoijan, P., Olek, A., and Piepenbrock, C., Bioinformatics. 17 Suppl 1:157-164, 2001).

Thus, step 4 provides for comparing, among a plurality of test genomic DNA samples corresponding to different test tissues and/or subjects, and using, preferably, at least one of a medium- or a high-throughput controlled assay suitable therefore, the methylation states corresponding to the secondary differentially methylated CpG dinucleotide sequence, or to the pattern, whereby a reliable methylation marker is provided.

Step 5(optional)—Ranking of CpG Positions:

In a preferred embodiment of the method an additional Step 5 is carried out that consists of ranking the CpG positions identified according to their capability of distinguishing between said classes of biological samples. Those CpG positions, which show the most significant methylation status differences between said classes are combined to form a panel. Once the panel is defined algorithmic methods for the classification of a sample based on the methylation status of the CpG positions within the panel are developed.

Step 6 (optional)—Panel Validation:

In a particularly preferred embodiment, the identified and selected CpG marker positions are further utilized in the design of an applied assay suitable for commercial clinical, diagnostic, research and/or high throughput application. Said applied assay may also be used to further validate the panel upon a larger sample set.

Several methods for the high throughput analysis of methylation within genomic DNA are available. These include restriction enzyme based analysis systems and more preferably bisulphite based methodologies such as Ms-SNuPE, hybridization analysis, MSP, and real-time PCR based applications. Once a suitable diagnostic assay has been assembled, the gene panel is validated by analysis of a test run of samples numbering in the hundreds. A diagnostic assay is understood to have been validated if it performs to the required levels of sensitivity and specificity, typically this would be a minimum sensitivity of 75%, and a minimum specificity of 90%.

Preferred methods for use in a diagnostic and/or prognostic applied assays comprise bisulfite treatment of the genomic DNA, followed by a primer and/or probe based detection methodology.

Particularly preferred embodiments comprise the use of MSP, MS-SNuPE, oligonucleotide hybridization (as described in Step 4 herein), MethyLight™ or HeavyMethyl™ assays, or combinations thereof.

Fluorescence-based Real-Time Quantitative PCR, and MethylLight™ assay. A particularly preferred embodiment comprises use of fluorescence-based Real-Time Quantitative PCR (Heid et al., Genome Res. 6:986-994, 1996) employing a dual-labeled fluorescent oligonucleotide probe (TaqMan™ PCR, using an ABI Prism 7700 Sequence Detection System, Perkin Elmer Applied Biosystems, Foster City, Calif.). The TaqMan™ PCR reaction employs the use of a non-extendible interrogating oligonucleotide, called a TaqMan™ probe, which is designed to hybridize to a GpC-rich sequence located between the forward and reverse amplification primers. The TaqMan™ probe further comprises a fluorescent “reporter moiety” and a “quencher moiety” covalently bound to linker moieties (e.g., phosphoramidites) attached to the nucleotides of the TaqMan™ oligonucleotide. For analysis of methylation within nucleic acids subsequent to bisulphite treatment, the probe is preferably methylation specific, as described in U.S. Pat. No. 6,331,393, (hereby incorporated by reference) also known as the MethylLight™ assay. Variations on the TaqMan™ detection methodology that are also suitable for use with the described invention include the use of dual probe technology (Lightcycler™) or fluorescent amplification primers (Sunrise™ technology). Both these techniques may be adapted in a manner suitable for use with bisulphite treated DNA, and moreover for inventive methylation analysis of CpG dinucleotides.

HeavyMethyl™. A further suitable method for assessment of methylation by analysis of bisulphite treated nucleic acids comprises the use of blocker oligonucleotides. The general use of such oligonucleotides has been described by Yu et al., BioTechniques 23:714-720, 1997. Blocking probe oligonucleotides are hybridized to the bisulphate-treated nucleic acid concurrently with the PCR primers. PCR amplification of the nucleic acid is terminated at the 5′ position of the blocking probe, thereby amplification of a nucleic acid is suppressed wherein the complementary sequence to the blocking probe is present. The probes may be designed to hybridize to the bisulphite-treated nucleic acid in a methylation status specific manner. For example, for detection of methylated nucleic acids within a population of unmethylated nucleic acids, suppression of the amplification of nucleic acids that are unmethylated at the position in question would be carried out by the use of blocking probes comprising a ‘CpG’ at the position in question, as opposed to a ‘CpA’ dinucleotide sequence, such as has been described in the German patent application DE 101 12 515.

MS-SNuP. In a further preferred embodiment, the determination of the methylation status of the CpG positions comprises use of template-directed oligonucleotide extension, such as “Ms-SNuPE” (Methylation-sensitive Single Nucleotide Primer Extension), described by Gonzalgo &Jones, Nucleic Acids Res. 25:2529-2531, 1997.

MSP. MSP (Methylation-specific PCR) refers to the art-recognized methylation assay described by Herman et al. Proc. Natl. Acad. Sci. USA 93:9821-9826, 1996, and by U.S. Pat. No. 5,786,146. In MSP applications, the use of methylation status specific primers for the amplification of bisulphate-treated DNA allows for distinguishing between methylated and unmethylated nucleic acids. MSP primer pairs contain at least one primer which hybridizes to a bisulphite-treated CpG dinucleotide of a pre-specified methylation state. Therefore, the sequence of said primers comprises at least one CpG, TpG or CpA dinucleotide. MSP primers specific for non-methylated DNA contain a ‘T’ at the 3′ position of the C-position in the CpG dinucleotide. Detection of the amplificate allows for the determination of the presence of a methylated nucleic acid. The use of MSP thereby allows for the detection of a nucleic acid of a pre-specified methylation state to be amplified against a background of alternatively methylated nucleic acids.

It is a preferred embodiment that said assay developed in step 6 of said method comprises the following steps:
a) treatment of the DNA such that all umnethylated cytosine bases are converted to uracil and wherein 5-methylcytosine bases remain unconverted,
b) amplification of one or more nucleic acid fragments comprising of one or more CpG positions identified in the marker identification step (step 4) of said method by means of at least two primer oligonucleotides,
c) detection of the amplified nucleic acids and concluding upon the methylation state of said CpG positions,
d) classification of the sample into one of said classes as defined in first step of said method.

In a particularly preferred embodiment, the treatment of step a) is carried out by means of chemical treatment, most preferably by means of treatment with a solution of bisulfite. It is preferred that the DNA is embedded in agarose before said treatment to keep the DNA in the single-stranded state during treatment, or, by treatment in the presence of a radical trap and a denaturing reagent, preferably an oligoethylene glycol dialkyl ether or, for example, dioxane. Prior to the PCR reaction, the reagents are removed either by washing in the case of the agarose method, or by standard art recognized DNA purification methods (e.g., precipitation or binding to a solid phase, membrane) or, simply by diluting in a concentration range that does not significantly influence the PCR.

Preferably, said classes of biological samples are determined according to the differentiation states of the cells said samples consist of.

This includes a set of classes that cannot be distinguished from each other with any technique currently available, other than methylation analysis or genome analysis. For example, it might not be possible to currently distinguish between a sample of a cell culture that has been treated with a certain agent 4 hour ago, and a sample of the same culture that has been treated with that same agent 8 hours ago. According to the particular definitions of ‘phenotypically distinct,’ these cells would not be phenotypically different. Therefore, it is also a preferred embodiment, and encompassed within the scope of the present invention, that said classes are composed of samples that are phenotypically identical, as in they are not phenotypically distinct samples. The two classes of samples are, nonetheless, according to the present invention, distinguishable by virtue of their differential methylation patterns.

In one embodiment of the method, said classes differ in that their biological samples are phenotypically distinct from one another. According to the definition of phenotypically distinct given herein, this would include cells that can be distinguished in other ways than by their methylation status or by genotypical differences.

Preferably, said biological samples are distinguishable by at least one suitable biochemical and/or histochemical marker.

Preferably, said classes differ in the age of said biological samples.

Preferably, said classes of biological samples differ in the specific time period that passed after a defined starting time point. The method can be used to determine which effect the passing time (under otherwise identical conditions) has on the methylation pattern of the biological sample of interest.

It is also important to determine whether classes of samples derived from different cell lines (may these be different in their origin or different in their culturing conditions) differ in said samples methylation pattern. If so, based on this methylation pattern analysis the method allows for classification of a biological sample in one of the classes accordingly. It is therefore an especially preferred embodiment of said method that said at least two classes are determined by at least two different cell lines said samples are derived therefrom.

Furthermore, it is preferred that said classes are determined by the different tissues and tissue-types said samples are derived from.

Said method can be used to distinguish between cells that are still differentiating further, and cells that are fully-differentiated. Therefore it is an especially preferred embodiment of said method that one of said two classes of biological samples is characterized by containing biological samples that consist of progenitor cells and the other class is characterized by containing differentiated cells derived from said progenitor cells.

It is especially preferred that said progenitor cell is a stem cell. Preferably, said stem cell is an adult stem cell. Furthermore, it is preferred that said progenitor cell is an adult stem cell.

Said method is, for example, used to classify β-cells. It is therefore especially preferred that said differentiated cell, derived from said progenitor cell, is a β-cell.

Preferbly, said biological samples consist of cells taken at several differentiation stages of progenitor cells developing into β-cells.

The present invention provides a method to distinguish β-cells as to whether they produce insulin or not, or furthermore, as to whether they do so in a glucose-responsive manner. To identify the relevant marker for this purpose, in step 1 the classes need to be determined accordingly. Therefore it is important that said classes contain biological samples that are characteristic for β-cells, which produce insulin and β-cells which do not.

It is especially preferred according to this invention that one class of biological samples consists of β-cells, which produce insulin and at least one other class of biological samples consists of β-cells, which do not produce insulin.

It is also especially preferred that one class of biological samples consists of β-cells, which produce insulin in a glucose-responsive manner and at least one other class of biological samples consists of β-cells, which do not produce insulin in a glucose-responsive manner.

Wherein said method is used to distinguish between distinct differentiation states of cells comprising said biological sample, and wherein one of said classes consists of progenitor cells, it is preferred that said progenitor cell belongs to a group comprising haematopoietic progenitor cells, myeloid progenitor cells, lymphoid progenitor cells, neural progenitor cells, mesenchymal progenitor cells, a progenitor cell isolated from a stromal vascular cell fraction of processed lipo-aspirate and nestin-positive pancreatic progenitor cells.

It is another preferred embodiment of said method, wherein said progenitor cell belongs to a group comprising of diploid liver cells, basal cells of epidermis, basal cells of nail bed, hair matrix cells, basal cells of epithelia, skeletal muscle satellite cells and osteoprogenitor cells.

The inventive method is also useful to differentiate between several origins of a cell, for example, the methylation patterns may differ between cells derived from an in vitro culture those derived from an in vivo source. It is therefore preferred that said method is used for identification of a tissue's cell of origin.

Several different sources of cells are identifiable based on the described method. It is preferred that said cells, which said biological samples are comprised of, are derived from in vitro cell cultures.

However it is also especially preferred that said cells are taken from biopsies and autopsies and/or said cells are taken from cell cultures derived from such in vivo and ex vivo sources.

It is particularly preferred that said method is used to ensure that an engineered cell tissue is derived from a specifically defined cell source.

Preferably, the inventive method is useful to distinguish cell lines being derived from in vitro sources from cell lines being derived from in vivo and/or autopsy sources.

The method described herein is a versatile method to classify different biological samples. A selection of specified uses of this method is given now.

The following are preferred uses of the present inventive method: monitoring cellular development or cellular differentiation; and improving the tissue engineering process.

To predict the successful differentiation of a cell, a set of marker genes is identified. Those marker genes do not need to significantly differ in their methylation states depending on the cells differentiation state but need to be differentially methylated depending on their ability to develop into a functioning cell. To identify those predictive marker genes, aliquots are be taken from a large number of cell cultures and stored until the cell's final fate can be determined. Specific differences in the methylation patterns can be associate with cells eventually developing into functioning cells, and with those that did not.

Knowing which methylation pattern indicates the potential failure of a cell to become a fully-functioning cell, will enable the selection for those cells that look promising. The earlier these sets of marker genes differ in their methylation pattern, the earlier those cells can be selected and the more efficient the process of cell culturing will be.

The use of said method for validation of engineered tissue cells is also preferred.

The use of said method for detecting contamination of differentiated cells or engineered tissue with progenitor cells is especially preferred.

Preferably the inventive method is used for distinguishing omnipotent cells from already differentiated cells.

Particularly preferred is the use of the inventive method for post-surgery evaluation of the development of tissue transplanted into a patient.

While the present invention has been described with specificity in accordance with certain of its preferred embodiments, the following example serves only to illustrate the invention and is not intended to limit the invention within the principles and scope of the broadest interpretations and equivalent configurations thereof. As used in this specification and the appended claims, the singular forms “a,” “an” and “the” include plural referents unless the content clearly dictates otherwise. The example described below is meant to explain and enable the invention.

### EXAMPLE 1

(Montitoring the Production of Tissue Engineered Cartilage)

Chondrocytes: Current practice for the production of tissue-engineered cartilage begins with a biopsy (taking cells from the patients cartilage tissue). The fresh biopsy material consists of fully-differentiated chondrocytes. While these fully-differentiated cells are not capable of expansion, they de-differentiate and start propagation (expansion), when cultured under two-dimensional tissue culture conditions. The expanded culture can be induced to re-differentiate by the supplementation of growth factors and provision of a three-dimensional matrix. The expanded and re-differentiated material is re-injected into the diseased area of the patient. For the patient's well being it is of utmost importance that the cells he or she receives are fully-differentiated and not able to de-differentiate in vivo.

To identify markers suitable to distinguish de-differentiated and expanding chondrocytes from fully-differentiated cartilage tissue, the following steps are performed according to the invention:

Step 1: The question to be addressed is “Is it possible to distinguish between chondrocyte cells that are completely differentiated and growth inhibited from chondrocytes that are de-differentiated and expanding?”. Accordingly, the two different classes of biological samples are class 1: differentiated chondrocyte cells are taken from healthy cartilage tissue of (at least three) individuals without known history of joint problems (from a tissue library), and class 2: chondrocyte precursor cells are taken from in vitro cartilage cell cultures that de-differentiated from chondrocyte cells (Jakob et al., J. Cell Biochem. 81:368-77, 2001).

Chondrocyte cells are isolated by incubating the cartilage tissue sample for a period of 22 hours at 37° C. in 0.15% type II collagenase, resuspending it in Dulbeccos modified Eagle medium (BMEM: detailed information about it can be found for example at: http://methdb.igh.cnrs.fr/cgrunau/cell_lines/DMEM.pdf).

The genomic DNA from both sample types of chondrocytes is isolated and purified according to the manufacturers guidelines given in the QIAamp™ DNA minikit, or according to the techniques described in the art.

Step 2: To identify differentially methylated CpG positions so called ‘Methylated Sequence Tags’ a combination of two techniques (DMH and RLGS) is performed.

First CpG island tags are generated as described (in Huang et al., Hum. Mol. Genet. 8:459-470, 1999) to create a CGI library, which is then arrayed onto a glass slide.

For this purpose, genomic DNA from the same source as the chondrocyte cells (same patient or same cell line) is isolated, purified and digested by the restriction enzyme MseI. The DNA digest is then enriched for CpG rich regions. For this purpose the digest is in vitro methylated and purified using a methylated DNA binding column consisting of a polypeptide of the DNA binding domain of the rat MeCP2 protein attached to a solid support (Cross et. al., Nature Genetics 6:236-244, 1994). The restriction fragments are screened for repeat elements and PCR amplified. The fragments are then fixed in the form of an array on a glass slide, in a manner whereby each fragment is locatable and identifiable on the surface.

Next, amplicons are prepared, representing a pool DNA from the genomes of the two classes of chondrocyte cells: Genomic DNA from both, the differentiated cells and the precursor cell samples are isolated. Each DNA sample is digested by MseI. To the ends of these DNA fragments linker sequences are ligated. The DNA fragments are then hydrolytically cleaved, catalyzed by two methylation sensitive restriction enzymes.

Said amplicons (the digest fragments) are now PCR amplified (the PCR primers binding to the linker sequences) and labeled to be used as probes in array-hybridization. Where the restriction of a fragment has taken place during the second digest, no PCR amplificate is detectable. Therefore, positive signals identified by the amplicons representing the differentiated-cell-class, but not by the amplicons representing the precursor-cell-class, indicate the presence of hypermethylated CpG island loci in chondrocyte cells of the sample representing the differentiated cell class.

Finally, the labeled PCR products are hybridized to the CGI library generated earlier. By comparison of the hybridization pattern of PCR fragments from said classes of chondrocytes, differences in methylation patterns between the two types of cells become apparent.

In a second experiment differentially methylated CpG positions are identified by generating RLGS profiles of said two classes of cells. Genomic DNA is extracted from both classes as described above using standard methods known in the art (e.g., by use of commercially available kits). Each sample is treated (cleaved ends and nicks and gaps are filled with nucleotide analogues) in order to prevent random labeling of the DNA strands in the next step. Each sample is treated (cleaved ends and nicks and gaps are filled with nucleotide analogues) to prevent random labeling of the DNA strands. Blocking the random (sheared) ends of the whole genomic DNA in the initial DNA preparations for RLGS include the addition of modified nucleotide bases to overhanging ends, where the newly added nucleotides prevent addition of other bases (radio-labeled nucleotides) in later steps. The modified nucleotides are a mixture of dideoxy-ATP, dideoxy-dTTp, dGTP-alpha-S & dCTP-alpha-S. The nucleotides are added to the overhanging ends with standard techniques using either DNA Polymerase 1 or Klenow enzyme (see e.g., Hatada et al., Proc Natl Acad Sci USA 88:9523-7, 1991).

The treated DNA is digested using the methylation-sensitive restriction enzyme NotI. The restriction enzyme is deactivated and the digest fragments are labeled at the restriction site by filling the NotI overhangs with radio-labeled dCTPs and dGTPs. The genomic DNA is further fragmented, with restriction endonucleases (e.g., EcoRV) with sequence recognition specificity that does not recognize sequences containing CpG, to separate the CpG islands.

The digest fragments are then separated by size, using high-resolution gel electrophoresis. The nucleic acid fragments are subjected to a restriction enzyme digest carried out in the gel. After digestion, the fragments are electrophoresed a second time with the current running perpendicular relative to the direction of the current in the first electrophoretic dimension. Thus, the digested fragments are separated in two dimensions.

Each gel is exposed to X-ray film to produce a fixed image of the positions of the fragment within the gel. These DNA fragment patterns on the X-ray films exposed to each of the 2-dimensional gels, are then compared to each other to determine where the patterns differ. Each missing spot represents a clone from the library and can be identified as such. A further analysis of those differentially methylated clones reveals the specifically differentially methylated CpG positions.

Step 3: The identified CpG positions are scored to select the most promising candidate CpG marker positions for further analysis in the next step. In this example, CpG positions that could be identified by both methods scored higher than those CpGs identified by one method only.

Step 4: The number of analyzed samples is increased to determine the identity of those CpG positions best suited for use as specific markers for one or the other class. Increase numbers of different cells are analyzed to get data that can eventually be evaluated by statistical means.

From those samples, the genomic DNA is isolated, purified and digested with MssI. Digested DNA is treated with bisulfite as described (Olek A, Oswald J and Walter J., Nucleic Acids Res. 24:5064-66, 1996).

The bisulfite-treated and successfully converted DNA is amplified via PCR using a specifically improved oligonucleotide-design method (see Clark & Frommer, In Taylor, G. R. (ed.) Laboratory Methods for the detection of Mutations and Polymorphisms in DNA. CRC Press, Boca Raton, Fla., pp 151-61, 1997).

Oligonucleotides with a C6-amino modification at the 5′-end are spotted with 4-fold redundancy on activated glass slides (Golub et al., Science 286:531-557, 1999). For each analyzed CpG position, two oligonucleotides-ne containing a CpG, the other one containing a TpG (reflecting the methylated and non-methylated status, respectively, of the CpG dinucleotides), are spotted and immobilized on the glass array.

Oligonucleotides are designed such that they match only the bisulfite-modified DNA fragments; this is, it is important to exclude signals arising from incomplete bisulfite conversion. The oligonucleotide microarrays representing up to 232 CpG sites are hybridized with a combination of up to 56 Cy5-labelled PCR fragments as described earlier (Chen D., et al., Nucleic Acids Res. 27:389-395, 1999). Subsequently, the fluorescent images of the hybridized slides are obtained using a GenePix™ 4000 microarray scanner (Axon Ilstruments). Hybridization experiments are repeated at least three times for each sample.

The CpG sites analyzed with the purpose of classifying the two classes of chondrocyte samples are located in the regulatory parts of one or several genes of the group comprising: Interleukin-1b, BMP-2/9, TGF-beta, FGF-2, Indian Hedgehog, Syndecan-3, PNCA, CollagenI/CollagenII, Aggrecan/CDRAP and Versican, Collagen XI, Collagen X, A-11, Viglin, COMB, TRAX/Translin, Matrilin-I, Fibromodulin, Epiphycan, Decorin, Biglycan, Sox-5, Sox-6, Sox-9, PTHrP, Chondroadherin, Annexin VI, Alkaline Phosphatase, GDF5, Noggin, Caspase3, Erk1/2. MEK/Erk, pMAPK38, Tyrosine Kinase, Vinculin, ID1, Cyclin D1, C-jun, JunD, and NFKB.

For class prediction (to differentiate between tissue development stages) a support vector machine (SVM) is used on a set of selected CpG sites. First the CpG sites for a given separation task are ranked by the significance of the difference between the two class means. The significance of each CpG is estimated by a two sample t-test. Then a SVM is trained on the most significant CpG positions, where the optimal number of CpG sites depends on the complexity of the separation task. The implementation of the SVM used the Sequential Minimal Optimization algorithm to find the 1-norm soft margin separating hyperplane (Christianini & Shawe-Taylor, (2000) An Introduction to Support Vector Machines and Other Kernel-Based Learning Methods. Cambridge University Press, Cambridge, UK, 2000).

To apply an additional independent data validation method, direct bisulfite sequencing reactions and/or Real-Time PCR are performed for those CpGs that seem to be significant, based on the interpretation of chip-based and statistical validation data.

The most significant CpGs found allow an unambiguous discrimination of completely differentiated and growth inhibited chondrocytes, and de-differentiated chondrocyte precursor cells.

1. A method for detecting and characterizing different developmental stages or differentiation of cells, comprising:
a) obtaining a set of at least two biological samples in each case having genomic DNA, wherein the biological samples correspond to at least two sample classes that are distinguishable by at least one of a phenotypic or measurable parameter;
b) identifying, using an assay suitable for comparing methylation status between or among corresponding CpG dinucleotide positions within the respective sample class genomic DNA samples, a plurality of primary differentially methylated CpG dinucletide sequence positions;
c) selecting at least one of the primary differentially methylated CpG dinucletide sequence positions, based on scoring thereof according to likely utility for discriminating between said at least two sample classes; and
d) confirming, as among a larger set of such biological samples, and using an assay suitable therefore, the class-distinguishing methylation status of at least one such selected primary differentially methylated CpG dinucleotide sequence position, whereby a reliable methylation marker is provided.

2. The method of claim 1, further comprising, prior to confirming in d), identifying within a context DNA region surrounding or including one of the primary differentially methylated CpG dincleotide positions, and using an assay or database suitable therefore, at least one secondary differentially methylated CpG dinucleotide sequence, and wherein confirming the class-distinguishing methylation status in d) further comprises confirming the class-distinguishing methylation status of the at least one secondary differentially methylated CpG dinucleotide sequence position.

3. The method of claim 1, further comprising, subsequent to confirming in d), raning the confirmed CpG positions according to their utility in distinguishing between said sample classes.

4. The method of any one of claims 1, 2 or 3, further comprising, in an additional step (e), developing an applied assay to determine the methylation status of the confirmed CpG positions in any biological sample.

5. The method of claim 4, wherein said applied assay comprises a methylation assay selected from the group consisting of MSP, MethyLight™, HeavyMethyl™, MS-SNuPE, and combinations thereof.

6. The method of claim 4, wherein said applied assay comprises:
i) treating of genomic DNA to convert all unmethylated cytosine bases to uracil, or to another base which is detectably dissimilar to cytosine in terms of hybridization properties, and wherein 5-methylcytosine bases remain unconverted;
ii) amplifying one or more of the CpG positions confirmed in d) using at least 2 primer oligonucleotides and a polymerase;
iii) detecting of the amplified nucleic acids;
iv) determining the methylation status of one or more CpG dinucleotide positions; and
v) classifying the sample into one of said classes.

7. The method of claim 6, wherein treating in i) comprises use of a bisulfite reagent.

8. The method of claim 1, wherein said assay suitable for comparing methylation status between or among corresponding CpG dinucleotide positions within the sample class genomic DNAs comprises a genome-wide assay or discovery technique useful for simultaneously treating the whole genome, or a representative fraction thereof, wherein identification of differentially methylated CpG positions is independent of genomic location.

9. The method of claim 8, wherein said genome-wide assay or discovery technique is selected from the group consisting of: differential methylation hybridization (DMH); NotI restriction based differential methylation hybridization (NR-DMH); restriction landmark genomic scanning (RLGS); methylated CpG island amplification (MCA); arbitrarily primed polymerase chain reaction (AP-PCR); and combinations thereof.

10. The method of any one of the preceding claims, wherein said classes of biological samples are determined according to the differentiation states of the cells said samples consist of, or are derived from.

11. The method of any one of the preceding claims, wherein said classes differ in that their corresponding biological samples are phenotypically distinct from one another.

12. The method of claim 10, wherein said classes of biological samples consist of samples which are phenotypically identical to one another.

13. The method of claim 10, wherein said classes differ in the age of the corresponding biological samples.

14. The method of claim 10, wherein said classes are distinguishable by at least one of a suitable biochemical or histochemical assay.

15. The method of claim 10, wherein said classes of biological samples differ in the specific elapsed time-period subsequent to a defined starting time-point.

16. The method of claim 10, wherein said at least two classes are characterized by at least two different cell lines said samples are derived from.

17. The method of claim 10, wherein said classes are characterized by the different tissues and tissue-types said samples are derived from.

18. The method of claim 10, wherein one of said two classes is characterized by comprising biological samples that consist of progenitor cells, and the at least one other class is characterized by containing differentiated cells derived from said progenitor cells.

19. The method of claim 18, wherein said progenitor cells are stem cells.

20. The method of claim 18, wherein said progenitor cells are embryonic stem cells.

21. The method of claim 18, wherein said progenitor cells are adult stem cells.

22. The method of claim 18, wherein said progenitor cells are selected from the group consisting of haematopoietic progenitor cells, myeloid progenitor cells, lymphoid progenitor cells, neural progenitor cells, mesenchymal progenitor cells, progenitor cells isolated from a stromal vascular cell fraction of processed lipoaspirate, and nestin-positive pancreatic progenitor cells.

23. The method of claim 18, wherein said progenitor cells are selected from the group consisting of diploid liver cells, basal cells of epidermis, basal cells of nail bed, hair matrix cells, basal cells of epithelia, skeletal muscle satellite cells and osteoprogenitor cells.

24. The method of claim 18, wherein said differentiated cell is a 13-cell.

25. The method of claim 10, wherein said biological samples consist of cells taken at several differentiation stages of progenitor cells developing into β-cells.

26. The method of claim 11, wherein one class of biological samples consists of β-cells that produce insulin, and at least one other class of biological samples consists of β-cells that do not produce insulin.

27. The method of claim 11, wherein one class of biological samples consists of β-cells that produce insulin in a glucose-responsive manner, and at least one other class of biological samples consists of β-cells that produce insulin not in a glucose-responsive manner.

28. The method of claim 10, wherein said cells are derived from in vitro cell cultures.

29. The method of claim 10, wherein said cells are selected from the group consisting of: biopsies; autopsies; cell cultures derived from at least one of biopsies or autopsies; cell cultures derived from in vivo sources; and cell cultures derived from ex vivo sources.

30. Use of the method of any one of claims 1-29 for monitoring a cell development or cell differentiation process.

31. Use of the method of any one of claims 1-29 for validating engineered tissue cells.

32. Use of the method of any one of claims 1-29 for detecting contamination of differentiated cells or engineered tissue with progenitor cells.

33. Use of the method of any one of claims 1-29 for ensuring that an engineered cell tissue is derived from a specifically defined cell source.

34. Use of the method of any one of claims 1-29 for identifying a tissue's cell of origin.

35. Use of the method of any one of claims 1-29 for distinguishing cell lines derived from in vitro sources, from cell lines derived from at least one of in vivo or autopsy sources.

36. Use of the method of any one of claims 1-29 for distinguishing omnipotent cells from already differentiated cells.

37. Use of the method of any one of claims 1-29 for post-surgery evaluation of the development of tissue transplanted into a patient.

38. Use of the method of any one of claims 1-29 for improving the tissue engineering process.
